(19) United States
(12) Patent Application Publication
(10) Pub. No.: US 2015/0105778 A1
(43) Pub. Date: Apr. 16, 2015

(54) BONE FIXATION PLATE

(71) Applicant: National Cheng Kung University, Tainan City (TW)

(72) Inventors: Haw-yen CHIU, Tainan City (TW); Wei-jr Lin, Tainan City (TW); Ting-mao Chou, Tainan City (TW); Fong-chin Su, Tainan City (TW)

(73) Assignee: National Cheng Kung University, Tainan City (TW)

(21) Appl. No.: 14/106,769

(22) Filed: Dec. 15, 2013

(30) Foreign Application Priority Data
Oct. 15, 2013 (TW) 102137166

(51) Int. Cl. A61B 17/80

(52) U.S. Cl. CPC A61B 17/80
USPC 606/71

(57) ABSTRACT

A bone fixation plate for fixing a fracture part of a bone is disclosed and can stably fix on a surface of a bone. The bone fixation plate has a protruding curved face, an indentation curved face and two rows of through holes. The protruding curved face has a blank portion and two binding portions. A suture is retained in the through holes formed close to two end edges of the binding portions, so that the stability of the bone fixation plate is improved.

### FIELD OF THE INVENTION

The present invention relates to a bone fixation plate, and more particularly to a bone fixation plate for stably fixing and positioning a fracture part of a bone.

### BACKGROUND OF THE INVENTION

Typically, a bone fixation plate is used to fix a fracture part of a bone of a patient, so that the fracture part of the bone will not be fractured again after be fixed, in order to enhance the healing of the fracture part of the bone. For example, a bone fixation plate for fixing fractures in phalange is disclosed in U.S. Pat. No. 4,790,302 and comprises an indentation curved face, a protruding curved face and a plurality of holes, wherein the holes are penetrated through the indentation curved face and the protruding curved face. Accordingly, the protruding curved face is attached closely to the inner surface of the bone by fixing the bone fixation plate into the bone, and the bone fixation plate is then fastened by screw-connecting to the bone through the holes. The above-mentioned bone fixation plate is only used to fix on the inner surface of the bone by screws. If the bone fixation plate is used to fix on the outer surface of the bone by binding sutures, the suture-bound bone fixation plate may be still loosed or slidable because the sutures can not be stably fixed on the smooth outer surface of the bone fixation plate. As a result, the positioning effect of the bone fixation plate for fixing on the outer surface of the bone is not good. Furthermore, the bone fixation plate must be fastened by screws, so that not only the process of drilling the bone is complicated, but also the operation will take longer time. Thus, the efficiency of the fixation process may be reduced.

Referring to FIGS. 1 and 2, a bone fixation plate 1 is designed to solve the above problem, wherein the bone fixation plate 1 has an indentation curved face 11 and a protruding curved face 12 opposite to the indentation curved face 11, two long edges 13 and two short edges 14 are provided between the indentation curved face 11 and the protruding curved face 12, the protruding curved face 12 has a plurality of channels 15 between the two short edges 14, and two opposite ends of the channels 15 are extended to the two long edges 13, respectively.

However, because the channels 15 are disposed on the protruding curved face 12 of the bone fixation plate 1, the sutures are only placed in the channels 15, and the effect of retaining the sutures is thus poor. When the bone fixation plate 1 is shifted by motion, the sutures in the channels 15 are easily loosed, so that the stability of the bone fixation plate needs to be improved.

Therefore, an improved bone fixation plate needs to be provided to solve the problem of prior art.

### SUMMARY OF THE INVENTION

A primary object of the present invention is to provide a bone fixation plate, which provides two rows of through holes to bind a bone with sutures, so that the sutures can be stably retained in the through holes.

Another object of the present invention is to provide a bone fixation plate, which provides two notches, so that the bone fixation plate can be closely attached onto the bone, and the sutures are retained by retaining structures to avoid from loosing.

To achieve the above objects, the present invention provides a bone fixation plate for fixing a fracture part of a bone, which comprises: a protruding curved face having a blank portion and two binding portions, wherein the binding portions are extended from two opposite sides of the blank portion for winding sutures around the binding portions and the bone; an indentation curved face opposite to the protruding curved face for covering the fracture part and abutting against the bone; and two rows of through holes penetrated through the binding portions, wherein the two rows of through holes are located close to two opposite end edges of the binding portions, respectively.

In one embodiment of the present invention, the bone fixation plate further comprises at least two retaining structures, the retaining structures are channels separately formed on the two binding portions, and the two rows of the through holes are correspondingly communicated with two ends of one of the channels.

In one embodiment of the present invention, the bone fixation plate further comprises two notches, and the notches are formed on two opposite end edges of the blank portion.

To achieve the above objects, the present invention provides another bone fixation plate for fixing a fracture part of a bone, which comprises a protruding curved face having a blank portion and two binding portions, wherein the binding portions are extended from two opposite sides of the blank portion for winding sutures around the binding portions and the bone; an indentation curved face opposite to the protruding curved face for covering the fracture part and abutting against the bone; at least two retaining structures separately formed on the two binding portions; and two notches formed on two opposite end edges of the blank portion.

In one embodiment of the present invention, the bone fixation plate further comprises two rows of through holes penetrated through the binding portions, and the two rows of the through holes are located close to two opposite end edges of the binding portions, respectively.

In one embodiment of the present invention, each of the binding portions is formed with a pair of the through holes, and the through holes are located on the two opposite end edges, respectively.

In one embodiment of the present invention, each of the binding portions is formed with two or more pairs of the through holes, and the through holes are located on the two opposite end edges of each of the binding portions, respectively, and arranged corresponding to the retaining structures.

In one embodiment of the present invention, the retaining structures are protrusions or channels.

In one embodiment of the present invention, the radius of curvature of the indentation curved face is 3 mm to 5 mm.

In one embodiment of the present invention, the length of the blank portion is equal to or smaller than one third of an axial length of the protruding curved face.

As described above, in the present invention, the sutures are not only wound/bound around the binding portions and the bone, but also the sutures are bound through the through holes and limited in the through holes, so that the stability of the bone fixation plate is improved. Furthermore, the notches are formed on two opposite end edges of the blank portion, so that the bone fixation plate can be closely attached onto the bone, and the sutures are retained by the retaining structures to avoid from loosing.

### DESCRIPTION OF THE DRAWINGS

FIGS. 1 and 2 are perspective views of a traditional bone fixation plate;

FIG. 3 is a perspective view of a bone fixation plate according to a first preferred embodiment of the present invention;

FIG. 4 is a perspective view of a bone fixation plate according to a second preferred embodiment of the present invention;

FIG. 5 is a perspective view of a bone fixation plate according to a third preferred embodiment of the present invention; and

FIG. 6 is a perspective view of a bone fixation plate according to a fourth preferred embodiment of the present invention.

### DETAILED DESCRIPTION OF THE PREFERRED EMBODIMENTS

The structure and the technical means adopted by the present invention to achieve the above and other objects can be best understood by referring to the following detailed description of the preferred embodiments and the accompanying drawings. Furthermore, directional terms described by the present invention, such as upper, lower, front, back, left, right, inner, outer, side, longitudinal/vertical, transverse/horizontal, and etc., are only directions by referring to the accompanying drawings, and thus the used directional terms are used to describe and understand the present invention, but the present invention is not limited thereto.

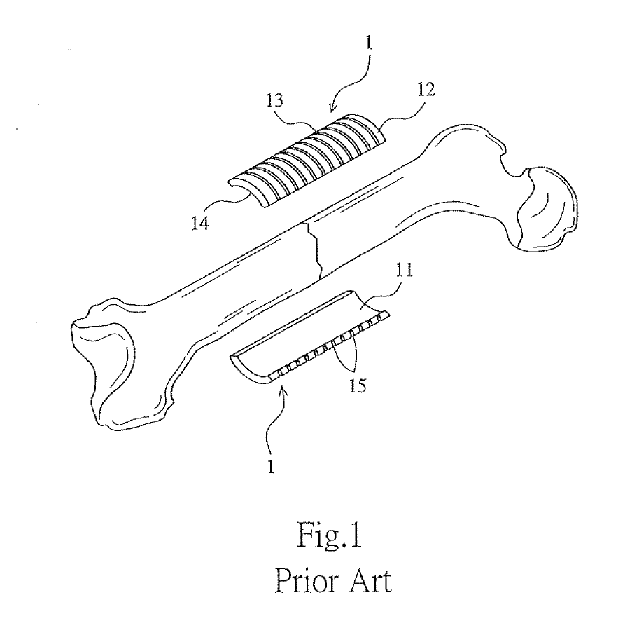

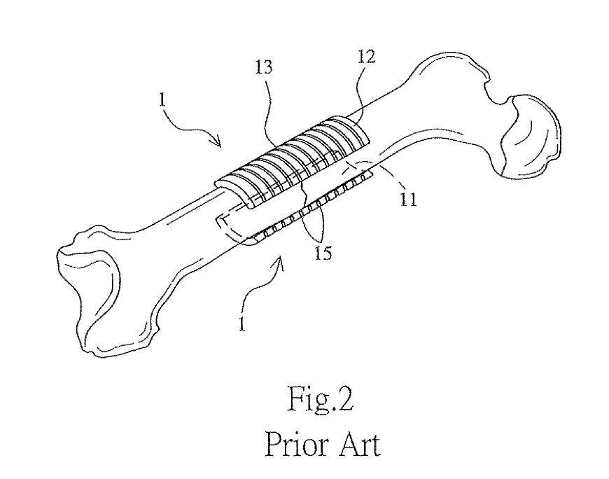

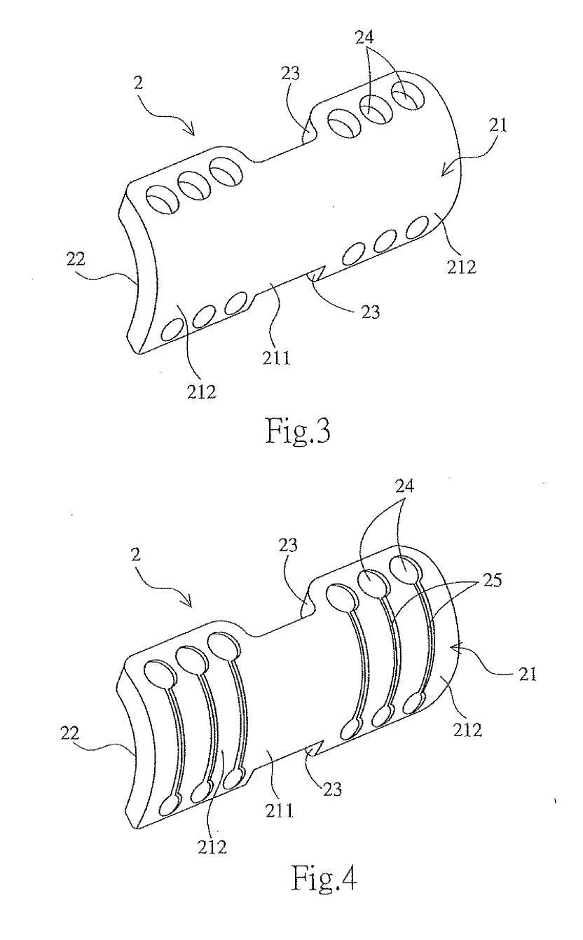

Referring now to FIG. 3, a bone fixation plate 2 according to a first preferred embodiment of the present invention is illustrated. The bone fixation plate 2 is an arc-shaped plate which has a protruding curved face and an indentation curved face, both of which are opposite to each other, wherein the bone fixation plate 2 is made of biocompatibility material, e.g. biodegradable material, stainless steel or titanium, which is fixed on an outer surface of a bone, as FIGS. 1 and 2, so that an injury or fracture portion of a bone of palm or other bone can be fixed by the aid of the bone fixation plate 2 to enhance the recovery thereof. The detailed structure of each component, assembly relationships and principle of operation in the present invention will be described in detail thereinafter.

Referring back to FIG. 3, the bone fixation plate 2 comprises a protruding curved face 21, an indentation curved face 22, two notches 23 and two rows of through holes 24, wherein the protruding curved face 21 is the outer surface of the bone fixation plate 2 for being bound by the sutures/wires as shown in FIGS. 1 and 2; the indentation curved face 22 is the inner surface of the bone fixation plate 2 and opposite to the protruding curved face 21 for attaching onto the bone; the protruding curved face 21 and the indentation curved face 22 are formed the arc-shaped plate, wherein the radius of curvature of the arc-shaped plate can be adjusted for different type of bones, the indentation curved face 22 is matched with a fracture part of the bone and attached to the bone by winding with sutures. In the embodiment, a bone of the palm is fixed by the bone fixation plate 2. The radius of curvature of the indentation curved face 22 is preferably 3 mm to 5 mm, but not limited thereto.

Referring to FIG. 3 again, the protruding curved face 21 has a blank portion 211 and two binding portions 212, wherein the binding portions 212 are extended from two opposite sides of the blank portion 211 for winding sutures around the binding portions 211 and the bone. In the embodiment of the present invention, preferably, an axial length of the protruding curved face 21 of the bone fixation plate 2 is 2 cm to 4 cm, and a length of each of the binding portions 211 is equal to or smaller than one third of the axial length of the protruding curved face 21.

Referring still to FIG. 3, the notches 23 are formed on two opposite end edges of the blank portion 211, and two rows of through holes 24 penetrated through the binding portions 212, wherein the two rows of through holes 24 are located close to two opposite end edges of the binding portions 212, respectively, and each row of through holes 24 are spaced equidistantly for the sutures to pass therethrough.

As described above, after a fracture part of a bone is treated, the bone fixation plate 2 according to the embodiment is used, and the fracture part of the bone is fixed by the bone fixation plate 2, wherein an appropriate radius of curvature of the bone fixation plate 2 must be pre-selected for matching with that of the fracture part of the bone. Furthermore, the amount of the bone fixation plate 2 is not limited, and can be one, two, three or more.

In the embodiment, two of the bone fixation plates 2 are used and placed on two opposite sides of the bone, respectively, and the bone fixation plates 2 are temporarily fixed by surgical instruments, so that the fracture part of the bone is commonly held and supported by two indentation curved faces 22 of the two bone fixation plates 2.

Then, the sutures are guided along the binding portion of one of the bone fixation plates 2 by surgical instruments, and then pass through the through holes 24 thereof, so that the sutures are wound around the protruding curved face 21 of the other bone fixation plate 2 and pass through the through holes 24 thereof, wherein the end of the sutures are fixed by surgical instruments, and the process of the winding is operated several times until the bone fixation plates 2 are stably attached onto the bone, so that the positioning of the bone fixation plates 2 on the fracture part of the bone is finished.

As described above, in the present invention, the sutures are not only wound around the binding portions 212 and the bone, but also the sutures are bound through the through holes 24 and limited in the through holes 24, so that the stability of the bone fixation plate 2 is improved. Furthermore, the notches 23 are formed on two opposite end edges of the blank portion 211, so that the bone fixation plate 2 is closely attached to the bone, and the sutures are retained by the retaining structures to avoid from loosing.

Referring to FIG. 4, a bone fixation plate according to a second embodiment of the present invention is illustrated and similar to the first embodiment, so that the second embodiment uses similar terms or numerals of the first embodiment. As shown, the difference of the second embodiment is that the bone fixation plate further comprises at least two retaining structures 25, the retaining structures 25 are recessed channels separately formed on the two binding portions 212, and the two rows of the through holes 24 are correspondingly communicated with two ends of one of the channels, so that the sutures are limited in the through holes 24 and channels.

As above, when the sutures are wound around the binding portions 212 and the bone, the sutures are limited in the through holes 24 and channels, so that the sutures are retained by the retaining structures to avoid from loosing, and the stability of the bone fixation plate 2 is improved.

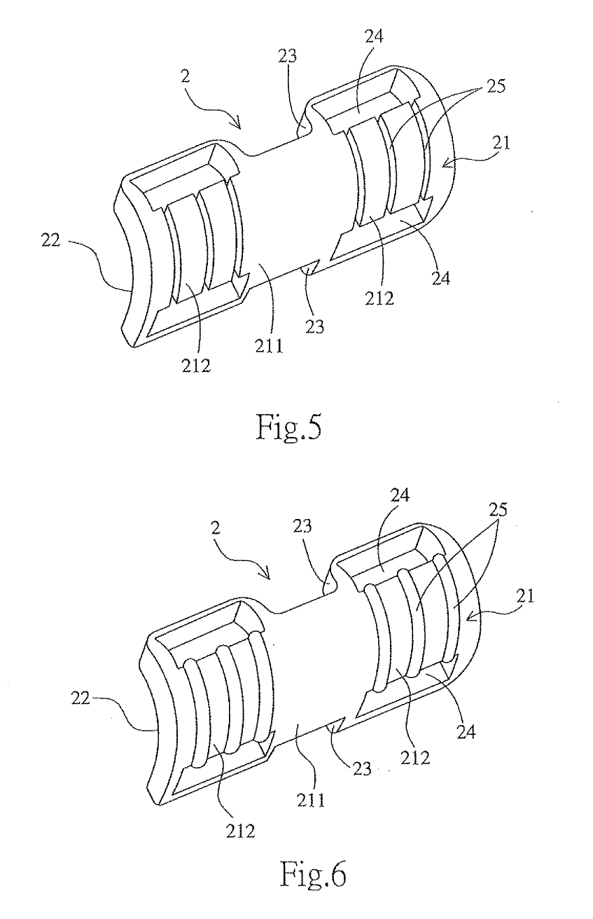

Referring to FIG. 5, a bone fixation plate according to a third embodiment of the present invention is illustrated and similar to the first embodiment, so that the third embodiment uses similar terms or numerals of the first embodiment. As shown, the difference of the third embodiment is that each of the binding portions 212 is only formed with a pair of the through holes 24, and the through holes 24 are located on the two opposite end edges, respectively. The retaining structures 25 are recessed channels separately formed on the two binding portions 212. When the sutures are wound around the binding portions 212 and the bone, the sutures are limited in the through holes 24 and channels, so that the sutures are retained by the retaining structures to avoid from loosing.

Referring to FIG. 6, a bone fixation plate according to a fourth embodiment of the present invention is illustrated and similar to the first embodiment, so that the fourth embodiment uses similar terms or numerals of the first embodiment. As shown, the difference of the fourth embodiment is that each of the binding portions 212 is formed with a pair of the through holes 24, the through holes 24 are located on the two opposite end edges, respectively, And the retaining structures 25 are protrusions separately formed on the two binding portions 212. When the sutures are around the binding portions 212 and the bone, the sutures are limited between two of the retaining structures 25 through the through holes 24, so that the sutures are retained by the retaining structures to avoid from loosing.

As described above, the sutures are wound around the binding portions 212 and the bone, the sutures pass through the two rows of through holes 24, so that the stability of the bone fixation plate 2 can be improved. The retaining structures 25 are protrusions or channels separately formed on the two binding portions 212 so that the sutures are retained/limited. Furthermore, the notches 23 are formed on two opposite end edges of the blank portion 211, so that the bone fixation plate 2 is closely attached to the bone.

The present invention has been described with a preferred embodiment thereof and it is understood that many changes and modifications to the described embodiment can be carried out without departing from the scope and the spirit of the invention that is intended to be limited only by the appended claims.

What is claimed is:

1. A bone fixation plate for fixing a fracture part of a bone, comprising:
a protruding curved face having a blank portion and two binding portions, wherein the binding portions are extended from two opposite sides of the blank portion for winding sutures around the binding portions and the bone;
an indentation curved face opposite to the protruding curved face for covering the fracture part and abutting against the bone; and
two rows of through holes penetrated through the binding portions, wherein the two rows of through holes are located close to two opposite end edges of the binding portions, respectively.

2. The bone fixation plate according to claim 1, wherein the bone fixation plate further comprises at least two retaining structures, the retaining structures are channels separately formed on the two binding portions, and the two rows of the through holes are correspondingly communicated with two ends of one of the channels.

3. The bone fixation plate according to claim 2, wherein the bone fixation plate further comprises two notches, and the notches are formed on two opposite end edges of the blank portion.

4. A bone fixation plate for fixing a fracture part of a bone, comprising:
a protruding curved face having a blank portion and two binding portions, wherein the binding portions are extended from two opposite sides of the blank portion for winding sutures around the binding portions and the bone;
an indentation curved face opposite to the protruding curved face for covering the fracture part and abutting against the bone;
at least two retaining structures separately formed on the two binding portions; and
two notches formed on two opposite end edges of the blank portion.

5. The bone fixation plate according to claim 4, wherein the bone fixation plate further comprises two rows of through holes penetrated through the binding portions, and the two rows of the through holes are located close to two opposite end edges of the binding portions, respectively.

6. The bone fixation plate according to claim 5, wherein each of the binding portions is formed with a pair of the through holes, and the through holes are located on the two opposite end edges, respectively.

7. The bone fixation plate according to claim 5, wherein each of the binding portions is formed with two or more pairs of the through holes, and the through holes are located on the two opposite end edges of each of the binding portions, respectively, and arranged corresponding to the retaining structures.

8. The bone fixation plate according to claim 4, wherein the retaining structures are protrusions or channels.

9. The bone fixation plate according to claim 4, wherein the radius of curvature of the indentation curved face is 3 mm to 5 mm.

10. The bone fixation plate according to claim 4, wherein the length of the blank portion is equal to or smaller than one third of an axial length of the protruding curved face.
